(19) United States
(12) Patent Application Publication
(10) Pub. No.: US 2021/0059712 A1
(43) Pub. Date: Mar. 4, 2021

(54) ASSEMBLY METHOD FOR TREATMENT INSTRUMENT AND RESIN PAD

(71) Applicant: OLYMPUS CORPORATION, Tokyo (JP)

(72) Inventor: Kazuhiro MORISAKI, Yokohama-shi (JP)

(73) Assignee: OLYMPUS CORPORATION, Tokyo (JP)

(21) Appl. No.: 17/097,331

(22) Filed: Nov. 13, 2020

Related U.S. Application Data
(63) Continuation of application No. PCT/JP2018/019893, filed on May 23, 2018.

(51) Int. Cl. A61B 17/32; B29C 65/00

(52) U.S. Cl. CPC A61B 17/320092; A61B 2017/00526; A61B 2017/320094; B29C 66/02

(57) ABSTRACT

An assembly method includes: inserting a resin pad including a pad body having a recessed portion, and a thick portion provided at a distal end portion of the recessed portion into a welding unit including a sheath and tape second gripping member assembled to the sheath along a groove provided in the second gripping member in a state where the recessed portion faces a first gripping member; inserting the ultrasonic probe into the welding unit; and forming a distal end of the resin pad into a claw shape by pressing the first gripping member against the thick portion by bringing the second gripping member and the first gripping member close to each other to form a depression having an inner surface shape complementary to an outer surface shape of the first gripping member at the thick portion.

This application is a continuation of International Application No. PCT/JP2018/019893, filed on May 23, 2018, the entire contents of which are incorporated herein by reference.

### BACKGROUND

The present disclosure relates to an assembly method for a treatment instrument and a resin pad.

In the related art, there is known a treatment instrument that treats a living tissue by applying treatment energy to the living tissue (see, for example, WO 2011/099571 A).

The treatment instrument described in WO 2011/099571 A employs ultrasonic energy as the treatment energy. Specifically, the treatment instrument includes first and second gripping members that grip the living tissue. The first gripping member is an ultrasonic probe that treats the living tissue by applying ultrasonic vibration to the living tissue. The second gripping member is configured to be openable and closable with respect to a distal end portion of the ultrasonic probe, and grips the living tissue with the distal end portion of the ultrasonic probe. Furthermore, in the second gripping member, on a side facing the distal end portion of the ultrasonic probe, a resin that comes into contact with the distal end portion when the second gripping member is brought closer to the distal end portion is provided.

Here, in the treatment instrument described in WO 2011/099571 A, a protruding portion that protrudes toward the ultrasonic probe is provided at a distal end portion of the resin pad in order to grip the living tissue reliably and prevent slipping when the living tissue is gripped. That is, the distal end portion of the resin pad is formed in a claw shape.

### SUMMARY

According to one aspect of the present disclosure, there is provided an assembly method for an ultrasonic treatment instrument including: a first gripping member including an ultrasonic probe configured to treat a living tissue; and a second gripping member configured to be opened and closed relative to the first gripping member, the assembly method including: inserting a resin pad including a pad body having a recessed portion, and a thick portion provided at a distal end portion of the recessed portion into a welding unit including a sheath and the second gripping member assembled to the sheath along a groove provided in the second gripping member in a state where the recessed portion faces the first gripping member; inserting the ultrasonic probe into the welding unit; and forming a distal end of the resin pad into a claw shape by pressing the first gripping member against the thick portion by bringing the second gripping member and the first gripping member close to each other to form a depression having an inner surface shape complementary to an outer surface shape of the first gripping member at the thick portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view illustrating a treatment instrument according to an embodiment;

FIG. 2 is a view illustrating a distal end portion of the treatment instrument;

FIG. 3 is a view illustrating a distal end portion of the treatment instrument;

FIG. 4 is a view illustrating a jaw;

FIG. 5 is a view illustrating the jaw;

FIG. 6 is a view illustrating an arm;

FIG. 7 is a view illustrating a wiper jaw;

FIG. 8 is a view illustrating the wiper jaw;

FIG. 9 is a view illustrating a resin pad;

FIG. 10 is a view illustrating the resin pad;

FIG. 11 is a view illustrating a mounting structure of the resin pad to the wiper jaw;

FIG. 12 is a view illustrating the mounting structure of the resin pad to the wiper jaw;

FIG. 13 is a flowchart indicating a reprocessing method for the treatment instrument;

FIG. 14 is a view for explaining Step S1;

FIG. 15 is a view for explaining Step S1;

FIG. 16 is a view for explaining Step S1;

FIG. 17A is a view for explaining Step S3;

FIG. 17B is a view for explaining Step S3; and

FIG. 18 is a view for explaining Step S3.

### DETAILED DESCRIPTION

Hereinafter, modes for carrying out the present disclosure (hereinafter, embodiments) will be described with reference to the drawings. The present disclosure is not limited to the embodiments described below. Further, in the description of the drawings, the same reference numerals are given to the same parts.

Schematic Configuration of Treatment Instrument

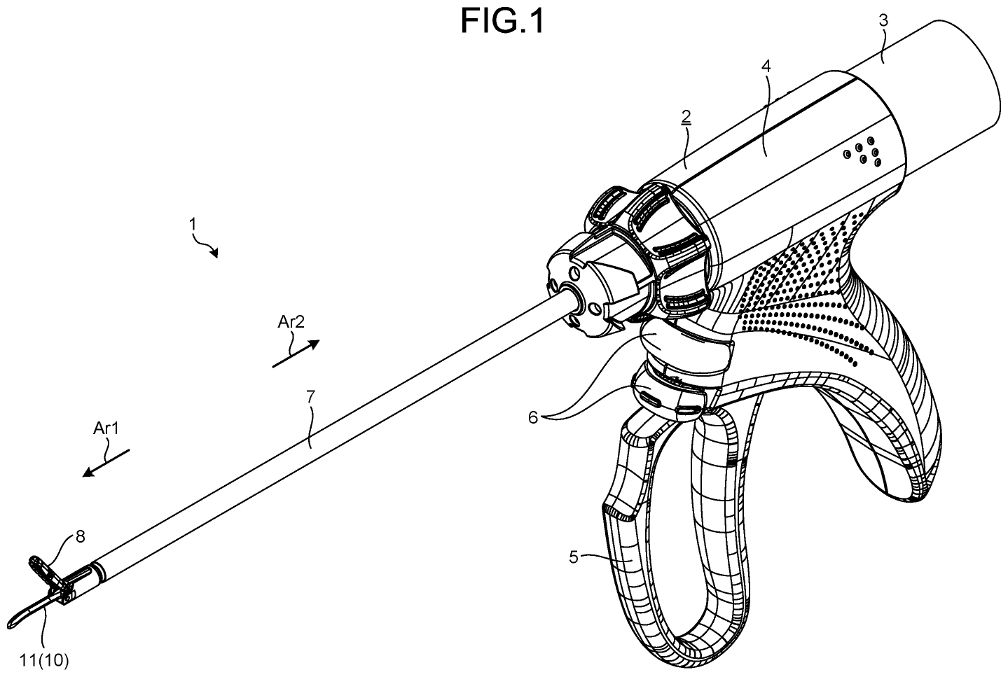

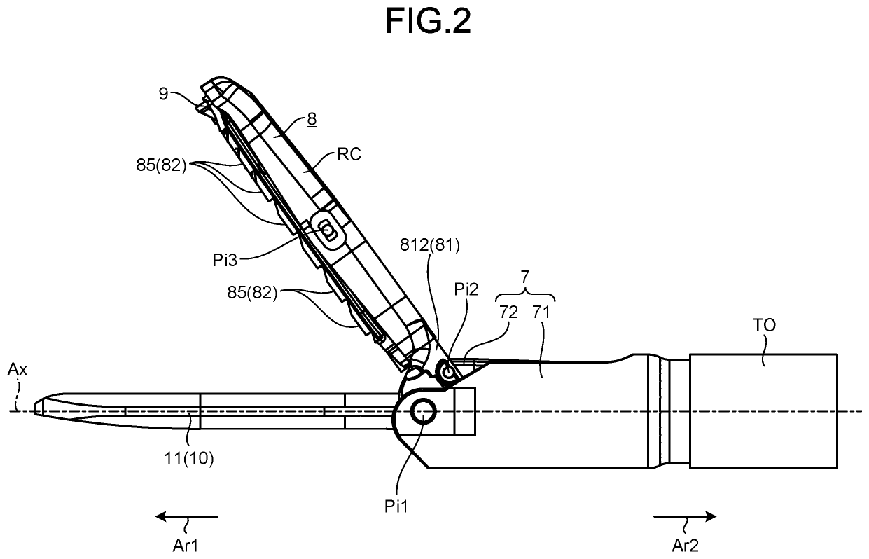

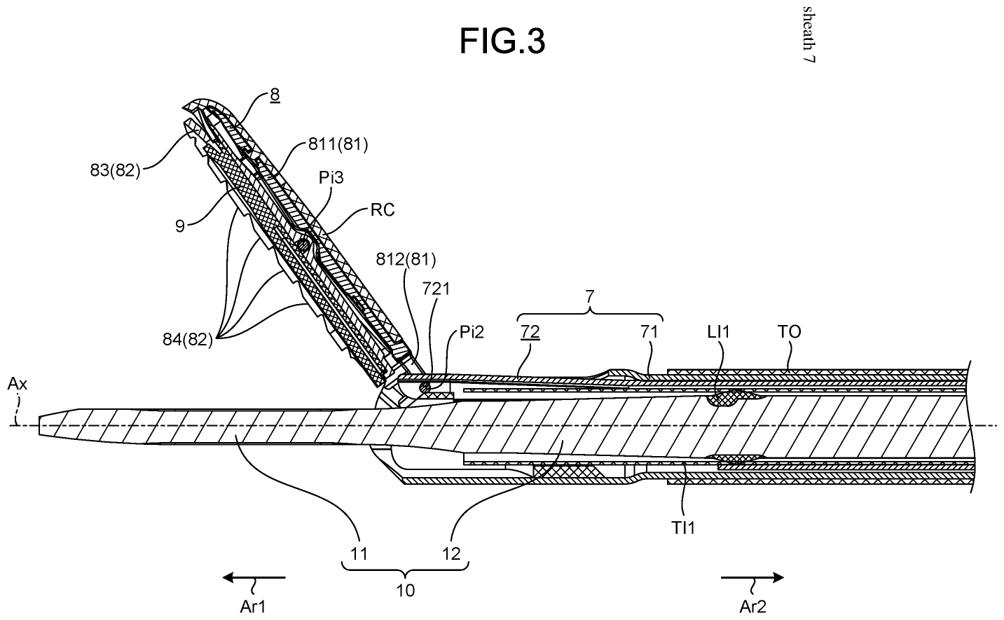

FIG. 1 is a view illustrating a treatment instrument 1 according to the present embodiment. FIGS. 2 and 3 are views illustrating a distal end portion of the treatment instrument 1. Specifically, FIG. 2 is a view seen from a direction orthogonal to the central axis Ax of a sheath 7. FIG. 3 is a cross-sectional view in which the distal end portion of the treatment instrument 1 is cut along a plane including the central axis Ax.

The treatment instrument 1 treats a site (hereinafter, referred to as a target portion) by applying ultrasonic energy and high-frequency energy to the target portion to be treated in a living tissue. Here, the treatment means, for example, coagulation and incision of a target portion. As illustrated in FIG. 1, the treatment instrument 1 includes a handpiece 2 and an ultrasonic transducer 3.

As illustrated in FIGS. 1 to 3, the handpiece 2 includes a holding case 4 (FIG. 1), an operation handle 5 (FIG. 1), switches 6 (FIG. 1), a sheath 7, a jaw 8, a resin pad 9 (FIGS. 2 and 3), and an ultrasonic probe 10.

The holding case 4 supports the entire treatment instrument 1. In the present embodiment, the holding case 4 is composed of two bodies. The holding case 4 is configured by combining the two bodies.

The operation handle 5 is movably mounted to the holding case 4 and receives an opening/closing operation by an operator.

The switches 6 are provided in a state of being exposed to the outside of the holding case 4, and receive an output start operation by the operator. Then, the switches 6 output an operation signal corresponding to the output start operation to a control device (not illustrated) electrically connected to the treatment instrument 1.

The sheath 7 has a substantially cylindrical shape as a whole. In the following, one side along the central axis Ax of the sheath 7 will be referred to as a distal end side Ar1 (FIGS. 1 to 3), and the other side will be referred to as a proximal end side Ar2 (FIGS. 1 to 3). Then, the sheath 7 is mounted to the holding case 4 by inserting a part of the proximal end side Ar2 into the holding case 4 from the distal end side Ar1 of the holding case 4. This sheath 7 includes an outer pipe 71 and an inner pipe 72, as illustrated in FIG. 2 or 3.

The outer pipe 71 is a cylindrical pipe made of a conductive material. The outer peripheral surface of the outer pipe 71 is covered with an electrically insulating outer tube TO (FIGS. 2 and 3). In the present embodiment, the outer tube TO is a heat-shrinkable tube and is held in close contact with the outer peripheral surface of the outer pipe 71 by heat shrinkage.

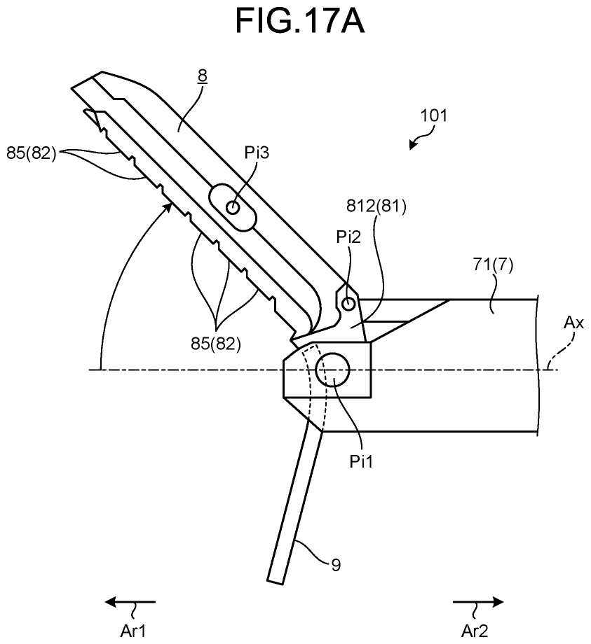

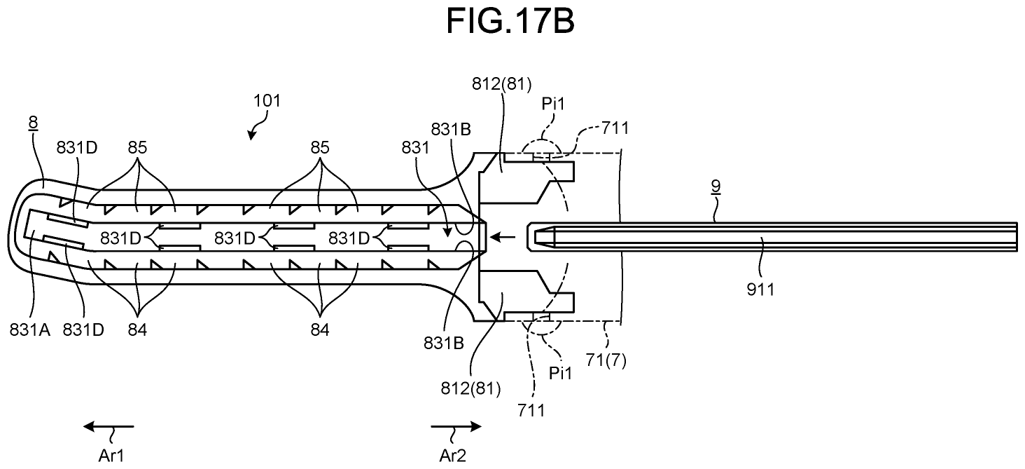

In this outer pipe 71, two first insertion holes 711 (see FIG. 17B) that penetrate through the inside and outside of the outer pipe 71 are provided at the end portion on the distal end side Ar1. A straight line connecting the two first insertion holes 711 intersects the central axis Ax and is orthogonal to the central axis Ax. Further, two first pins Pi1 (FIGS. 2 and 17B) are inserted into the two first insertion holes 711, respectively. In the present embodiment, the two first pins Pi1 are fixed to the outer pipe 71 by welding while being inserted into the respective first insertion holes 711.

The inner pipe 72 is a cylindrical pipe made of a conductive material and having a smaller diameter than the outer pipe 71. Further, the inner pipe 72 is inserted into the outer pipe 71 so as to be coaxial with the outer pipe 71. Then, the inner pipe 72 moves to the distal end side Ar1 or the proximal end side Ar2 along the central axis Ax according to the opening/closing operation of the operation handle 5 by the operator.

In this inner pipe 72, two second insertion holes 721 (FIG. 3) that penetrate through the inside and outside of the inner pipe 72 are provided at the end portion on the distal end side Ar1. In FIG. 3, only one of the two second insertion holes 721 is visible. The straight line connecting the two second insertion holes 721 is parallel to the straight line connecting the two first insertion holes 711 without intersecting the central axis Ax. Second pins Pi2 (FIG. 3) are inserted into the two second insertion holes 721.

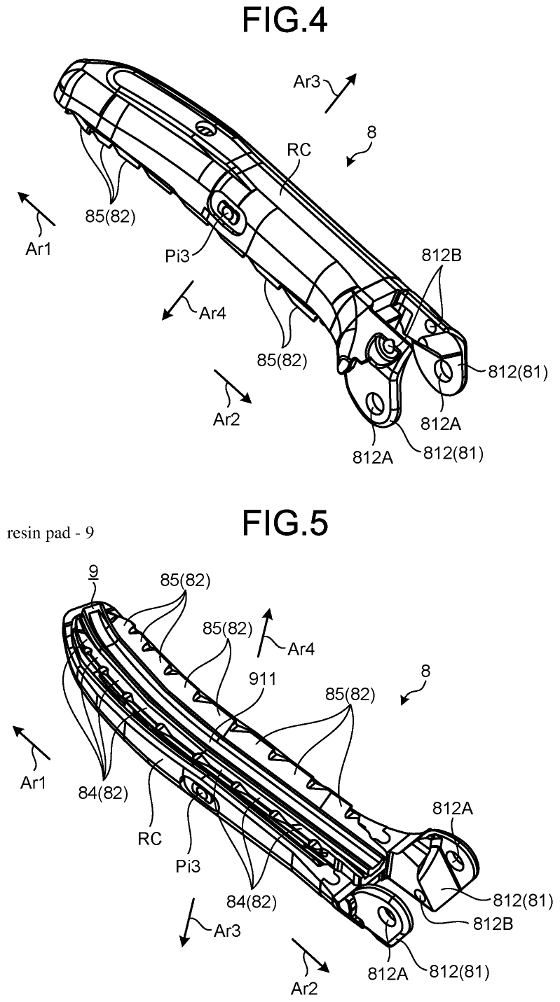

In the following, in describing the configurations of the jaw 8 and the resin pad 9, the side away from a blade 11 that constitutes the ultrasonic probe 10 is referred to as a back surface side Ar3 (see FIGS. 4 to 12), and the side approaching the blade 11 is referred to as a blade side Ar4 (see FIGS. 4 to 12).

FIGS. 4 and 5 are views illustrating the jaw 8. Specifically, FIG. 4 is a perspective view of the jaw 8 as seen from the back surface side Ar3. FIG. 5 is a perspective view of the jaw 8 as seen from the blade side Ar4. FIG. 5 illustrates a state in which the resin pad 9 is assembled to the jaw 8.

The jaw 8 corresponds to a second gripping member according to the present disclosure. The jaw 8 can be opened and closed with respect to the blade 11 (FIGS. 2 and 3) by being axially supported on the end portion of the sheath 7 on the distal end side Ar1. Then, the jaw 8 grips the target portion with the blade 11. This jaw 8 includes an arm 81 and a wiper jaw 82, as illustrated in FIG. 4 or 5.

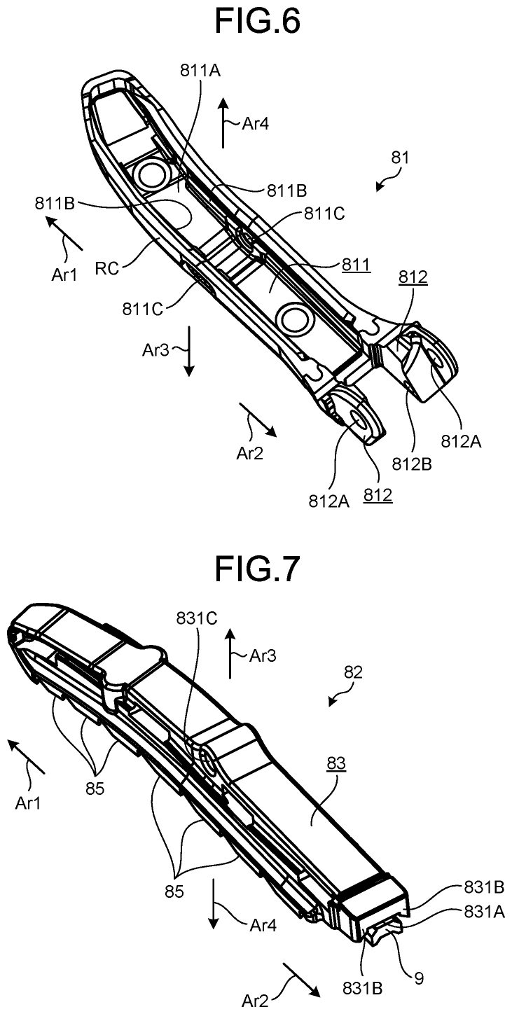

FIG. 6 is a view illustrating the arm 81. Specifically, FIG. 6 is a perspective view of the arm 81 as seen from the blade side Ar4.

The arm 81 is made of a conductive material. As illustrated in FIG. 6, the arm 81 is a member in which an arm body 811 and a pair of bearing portions 812 are integrally formed.

The arm body 811 is configured by an elongated plate body. In the present embodiment, the longitudinal direction of the arm body 811 is a direction along a curve that goes to the left side toward the distal end side Ar1 as seen from the proximal end side Ar2 in a state where the jaw 8 is located above the blade 11.

As illustrated in FIG. 6, in this arm body 811, the surface on the blade side Ar4 is provided with a first recessed portion 811A extending from the proximal end toward the distal end side Ar1 along the longitudinal direction of the arm body 811.

As illustrated in FIG. 6, in side wall portions 811B of the arm body 811 on both sides in the width direction that constitute the first recessed portion 811A, third insertion holes 811C that penetrate through the arm body 811 in the width direction and allow third pins Pi3 (FIGS. 4 and 5) to be inserted therethrough are provided, respectively. The two third insertion holes 811C are located at substantially the center of the arm body 811 in the longitudinal direction. In addition, the straight line connecting the two third insertion holes 811C is parallel to the width direction of the arm body 811. In the present embodiment, the third pins Pi3 are fixed to the arm body 811 by welding while being inserted into the respective third insertion holes 811C.

Further, an electrically insulating resin cover RC (FIGS. 4 to 6) is integrally formed on the surface on the back surface side Ar3 of the arm body 811 so as to cover the surface on the back surface side Ar3. In this embodiment, the resin cover RC is insert-molded on the arm body 811. However, the present disclosure is not limited to this. For example, a configuration in which the resin cover RC is fixed to the arm body 811 by a snap fit or a metal pin may be employed.

The pair of bearing portions 812 are each provided at the proximal end of the arm body 811 and are configured by plate bodies facing each other in the width direction of the arm body 811.

As illustrated in FIG. 6, the pair of bearing portions 812 are respectively provided with fourth insertion holes 812A that penetrate through the front and back sides and allow the two first pins Pi1 to be inserted therethrough. That is, the arm 81 is connected to the outer pipe 71 by the two first pins Pi1.

Further, as illustrated in FIG. 4, the pair of bearing portions 812 are respectively provided with fifth insertion holes 812B that penetrate through the front and back sides and allow the second pins Pi2 to be inserted therethrough. In the present embodiment, the second pins Pi2 are fixed to the arm 81 by welding while being inserted into the respective second insertion holes 721 and the respective fifth insertion holes 812B. That is, the arm 81 is connected to the inner pipe 72 by the second pins Pi2. Then, the arm 81 rotates about the two first pins Pi1 in conjunction with the movement of the inner pipe 72 to the distal end side Ar1 or the proximal end side Ar2 according to the opening/closing operation of the operation handle 5 by the operator. With this, the jaw 8 is opened and closed with respect to the blade 11.

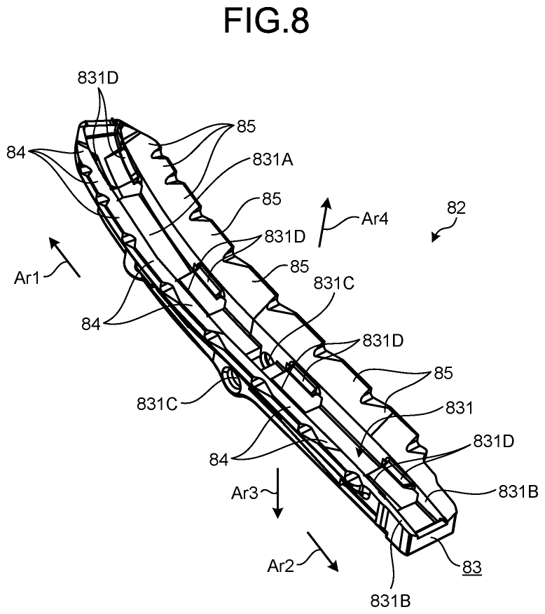

FIGS. 7 and 8 are views illustrating the wiper jaw 82. Specifically, FIG. 7 is a perspective view of the wiper jaw 82 as seen from the back surface side Ar3. FIG. 7 illustrates a state in which the resin pad 9 is assembled to the wiper jaw 82. FIG. 8 is a perspective view of the wiper jaw 82 as seen from the blade side Ar4.

The wiper jaw 82 is made of a conductive material and is mounted to the arm 81. As illustrated in FIG. 7 or 8, the wiper jaw 82 includes a wiper jaw body 83, a plurality of first tooth portions 84 (FIG. 8), and a plurality of second tooth portions 85.

The wiper jaw body 83 is configured by an elongated plate body extending along the longitudinal direction of the arm body 811. The outer shape of the wiper jaw body 83 is set to be substantially the same as the inner surface shape of the first recessed portion 811A. The wiper jaw body 83 is installed in the first recessed portion 811A.

As illustrated in FIG. 8, in this wiper jaw body 83, the surface on the blade side Ar4 is provided with a second recessed portion 831 penetrating from the proximal end to the distal end along the longitudinal direction of the wiper jaw body 83.

The second recessed portion 831 is a portion where the resin pad 9 is installed, and the resin pad 9 is in contact with a bottom surface 831A thereof. The bottom surface 831A comes into contact with the resin pad 9, and thus the end portion of the resin pad 9 on the distal end side Ar1 is set to be bent toward the blade side Ar4.

Further, as illustrated in FIG. 7 or FIG. 8, in side wall portions 831B on both sides of the wiper jaw body 83 in the width direction that constitute the second recessed portion 831, sixth insertion holes 831C that penetrate through the wiper jaw body 83 in the width direction and allow the third pins Pi3 to be inserted therethrough are provided, respectively. The two sixth insertion holes 831C are located at substantially the center of the wiper jaw body 83 in the longitudinal direction. In addition, the straight line connecting the two sixth insertion holes 831C is parallel to the width direction of the wiper jaw body 83. The wiper jaw body 83 is axially supported on the arm 81 so as to be swingable about the third pins Pi3. That is, the wiper jaw 82 is configured to be swingable about the third pins Pi3. Thus, the position at which the strongest force is applied to the target portion when the target portion is gripped between the jaw 8 and the blade 11 is set not on the proximal end side Ar2 of the jaw 8 but at the substantially center of the jaw 8 in the longitudinal direction. As a result, the force is applied to the target portion gripped between the jaw 8 and the blade 11 substantially uniformly.

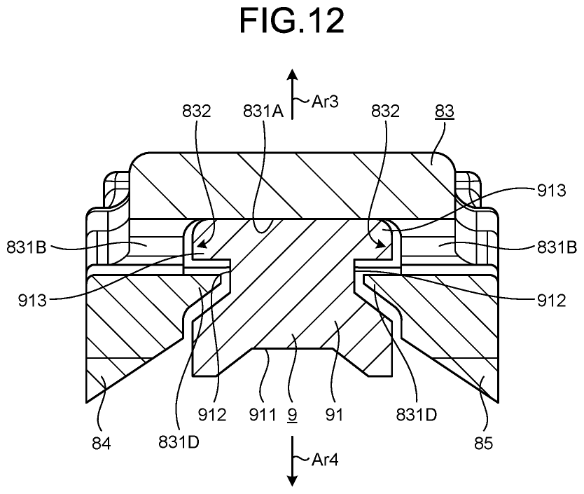

Further, as illustrated in FIG. 8, in the side wall portions 831B, a plurality of pairs (in the present embodiment, four sets) of claws 831D that protrude toward the inside of the second recessed portion 831 and face each other along the width direction of the wiper jaw body 83 are provided. The plurality of sets of claws 831D are arranged in parallel along the longitudinal direction of the wiper jaw body 83. The bottom surface 831A, the side wall portions 831B, and the claws 831D form grooves 832 (see FIG. 12) according to the present disclosure.

The plurality of first tooth portions 84 protrude from one side wall portion 831B toward the blade side Ar4 and are arranged in parallel along the longitudinal direction of the wiper jaw body 83.

The plurality of second tooth portions 85 protrude from the other side wall portion 831B toward the blade side Ar4 and are arranged in parallel along the longitudinal direction of the wiper jaw body 83.

The plurality of first tooth portions 84 and the plurality of second tooth portions 85 are provided with the resin pad 9 sandwiched therebetween in a state where the resin pad 9 is mounted to the wiper jaw 82.

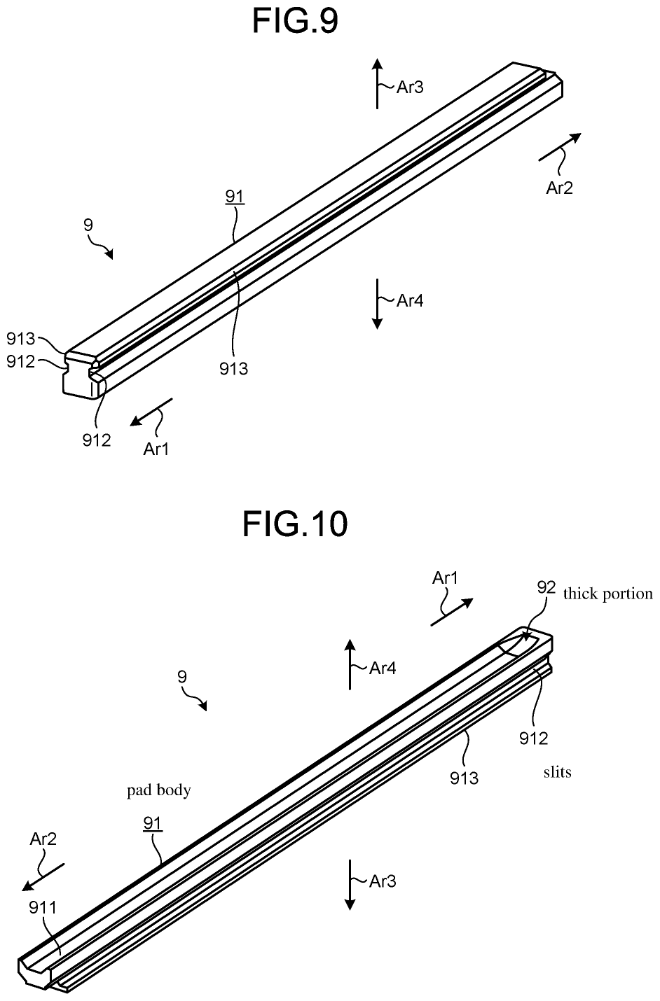

FIGS. 9 and 10 are views illustrating the resin pad 9. Specifically, FIG. 9 is a perspective view of the resin pad 9 as seen from the back surface side Ar3. FIG. 10 is a perspective view of the resin pad 9 as seen from the blade side Ar4. FIGS. 9 and 10 illustrate the resin pad 9 in a state before being remanufactured by a reprocessing method for the treatment instrument 1 described later.

The resin pad 9 is softer than the ultrasonic probe 10 and is made of a resin material having electrical insulation and biocompatibility, for example, polytetrafluoroethylene (PTFE). The resin pad 9 comes into contact with the blade 11 when the jaw 8 is brought close to the blade 11. As illustrated in FIG. 9 or 10, the resin pad 9 includes a pad body 91 and a thick portion 92 (FIG. 10).

The pad body 91 has a substantially rectangular parallelepiped shape that extends linearly.

As illustrated in FIG. 10, in the pad body 91, the surface of the blade side Ar4 is provided with a third recessed portion 911 extending from the proximal end toward the distal end Ar1.

Further, as illustrated in FIGS. 9 and 10, in the pad body 91, slits 912 penetrating from the distal end to the proximal end are provided on side surfaces intersecting the surface on the blade side Ar4. Here, of the side wall portions on both sides of the slit 912 in the width direction that form the slit 912, the side wall portion on the back surface side Ar3 corresponds to a protruding portion 913 (FIGS. 9 and 10) according to the present disclosure.

As illustrated in FIG. 10, the thick portion 92 is a surplus thickness provided on the distal end side Ar1 of the third recessed portion 911 in the pad body 91. Since the thick portion 92 is provided, the inner surface shape of the distal end side Ar1 in the third recessed portion 911 is not a shape complementary to the outer shape of the surface of the blade 11 that faces the jaw 8. The inner surface shape of the third recessed portion 911 other than the distal end side Ar1, that is, the inner surface shape of the portion of the third recessed portion 911 where the thick portion 92 is not provided is set to a shape complementary to the outer shape of the surface of the blade 11 that faces the jaw 8. That is, when the resin pad 9 is simply applied to the blade 11, a gap is formed between the inner surface of the third recessed portion 911 and the outer surface of the blade 11 due to the thick portion 92.

The inner surface shape of the portion of the third recessed portion 911 where the thick portion 92 is not provided may not be a complementary shape that completely matches the outer shape of the surface of the blade 11 that faces the jaw 8.

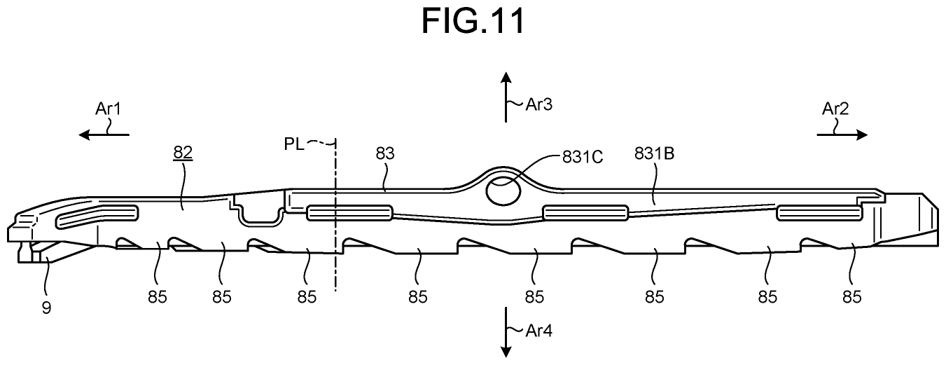

FIGS. 11 and 12 are views illustrating a mounting structure of the resin pad 9 to the wiper jaw 82. Specifically, FIG. 11 is a view of the wiper jaw 82 to which the resin pad 9 is mounted as seen from the width direction. FIG. 12 is a cross-sectional view of the wiper jaw 82 to which the resin pad 9 is mounted, taken along a plane PL (FIG. 11) orthogonal to the longitudinal direction.

As illustrated in FIG. 12, in the resin pad 9 described above, the pair of protruding portions 913 respectively enter the pair of grooves 832 of the wiper jaw 82 and are locked to the pair of claws 831D, so that the resin pad 9 is mounted to the wiper jaw 82. Then, the resin pad 9 is made attachable and detachable to and from the wiper jaw 82 by sliding the pair of protruding portions 913 along the longitudinal direction of the wiper jaw 82 in the pair of grooves 832.

The ultrasonic probe 10 corresponds to a first gripping member according to the present disclosure. The ultrasonic probe 10 has an elongated shape and is made of a conductive material. As illustrated in FIG. 2 or 3, the ultrasonic probe 10 is inserted into the inner pipe 72 with the blade 11 exposed to the outside. The ultrasonic probe 10 includes the blade 11 and a shaft 12.

The blade 11 is provided at the distal end of the shaft 12. Similar to the jaw 8, the blade 11 extends along a curve that goes to the left side toward the distal end side Ar1 as seen from the proximal end side Ar2 in a state where the jaw 8 is located above the blade 11.

The shaft 12 has an elongated shape extending along the central axis Ax, and the end portion on the proximal end side Ar2 is connected to a BLT (bolt-clamped Langevin type transducer) that constitutes the ultrasonic transducer 3. The shaft 12 transmits the ultrasonic vibration generated by the BLT to the blade 11 from the end portion on the proximal end side Ar2. In the present embodiment, the ultrasonic vibration is longitudinal vibration that vibrates in the direction along the central axis Ax. At this time, the blade 11 vibrates with a desired amplitude due to the longitudinal vibration of the ultrasonic probe 10.

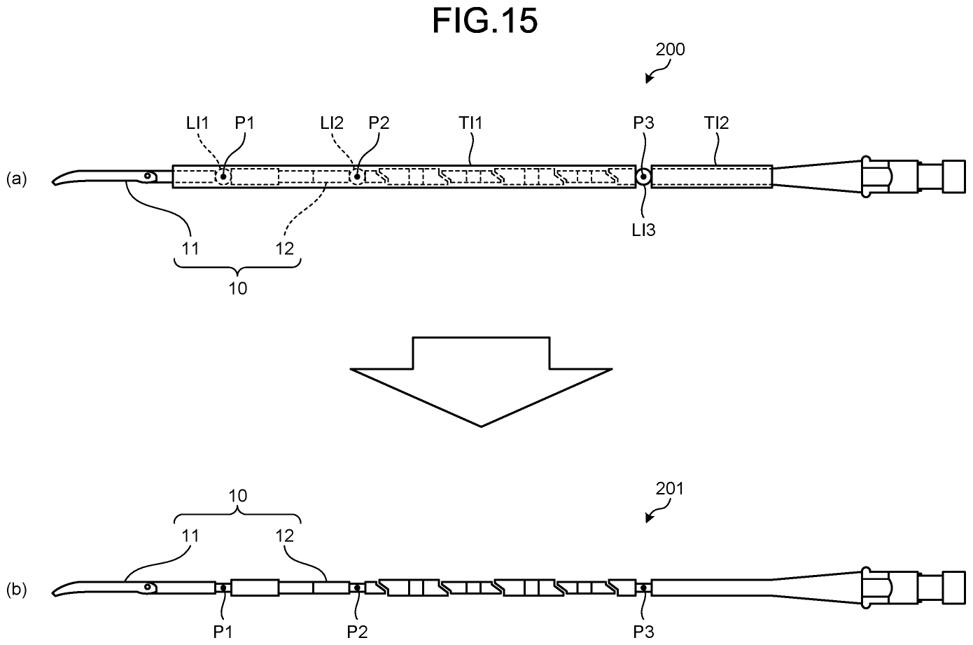

Annular first to third linings LI1 to LI3 (see FIG. 15) each having electrical insulation and elasticity, and extending along the circumferential direction around the central axis of the shaft 12 are mounted to the outer peripheral surface of the shaft 12. The first to third linings LI1 to LI3 are positioned at positions P1 to P3 (see FIG. 15) of nodes of the longitudinal vibration of the ultrasonic probe 10, respectively.

Further, the outer peripheral surface of the shaft 12 is covered with electrically insulating first and second inner tubes TI1 and TI2 (see FIG. 15).

The first inner tube TI1 covers the outer peripheral surface of the shaft 12 on the distal end side Ar1 while covering the first and second linings LI1 and LI2 provided on the distal end side Ar1 of the first to third linings LI1 to LI3.

The second inner tube TI2 covers, on the outer peripheral surface of the shaft 12, the proximal end side Ar2 with respect to the third lining LI3 provided on the most proximal end side Ar2 of the first to third linings LI1 to LI3. In the present embodiment, the second inner tube 112 is a heat-shrinkable tube and is held in close contact with the outer peripheral surface of the shaft 12 by heat shrinkage.

The first and second inner tubes TI1 and TI2 described above have a function of ensuring electrical insulation between the outer pipe 71 and the inner pipe 72 and the ultrasonic probe 10. Further, the first and second linings LI1 and LI2 have a function of sealing the liquid that has entered the gap between the first inner tube TI1 and the ultrasonic probe 10. In addition, the third lining LI3 has a function of sealing the liquid that has entered the gap between the inner pipe 72 and the first inner tube TI1.

The ultrasonic transducer 3 is detachably and attachably connected to the proximal end side Ar2 of the holding case 4. Although not specifically illustrated, the ultrasonic transducer 3 includes a BLT that generates ultrasonic vibration in response to supply of AC power.

The treatment instrument 1 described above operates as follows.

The operator holds the treatment instrument 1 by hand, and inserts the distal end portion of the treatment instrument 1 into the abdominal cavity through the abdominal wall using, for example, a trocar or the like. Then, the operator operates the operation handle 5 to open and close the jaw 8 with respect to the blade 11, so that the jaw 8 and the blade 11 grip the target portion. Then, the operator presses the switch 6. Then, a control device (not illustrated) electrically connected to the treatment instrument 1 executes the following control according to the operation signal from the switch 6.

The control device supplies a high-frequency current between the jaw 8 and the blade 11 via the outer pipe 71, the inner pipe 72, and the shaft 12. Specifically, the high-frequency current flows between the blade 11 and the plurality of first and second tooth portions 84 and 85 having the same electric potential. Then, the high-frequency current flows in the target portion gripped between the jaw 8 and the blade 11. In other words, high-frequency energy is applied to the target portion.

Further, the control device supplies to AC power to the BLT constituting the ultrasonic transducer 3 at substantially the same time as the supply of the high-frequency current between the jaw 8 and the blade 11, thereby generating ultrasonic vibration in the BLT. Then, the ultrasonic vibration is applied from the blade 11 to the target portion gripped between the jaw 8 and the blade 11. In other words, ultrasonic energy is applied to the target portion.

Then, Joule heat is generated in the target portion by the high-frequency current flowing therethrough. Further, due to the vertical vibration of the blade 11, frictional heat is generated between the blade 11 and the target portion. As a result, the target portion is incised while coagulating.

Reprocessing Method for Treatment Instrument

Next, the reprocessing method for the above-described treatment instrument 1 will be described.

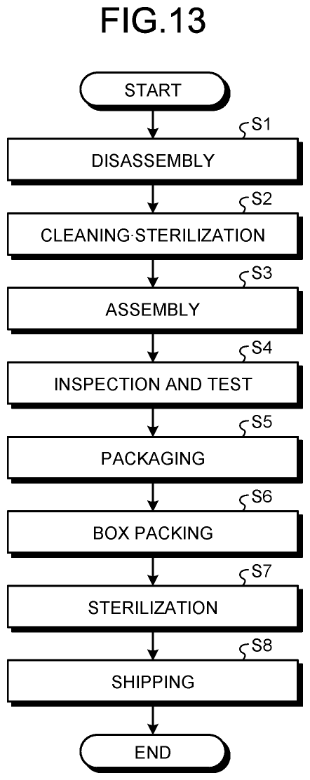

FIG. 13 is a flowchart indicating the reprocessing method for the treatment instrument 1.

The operator collects the used treatment instrument 1 after treating the target portion. Then, the operator disassembles the collected treatment instrument 1 as described below (Step S1).

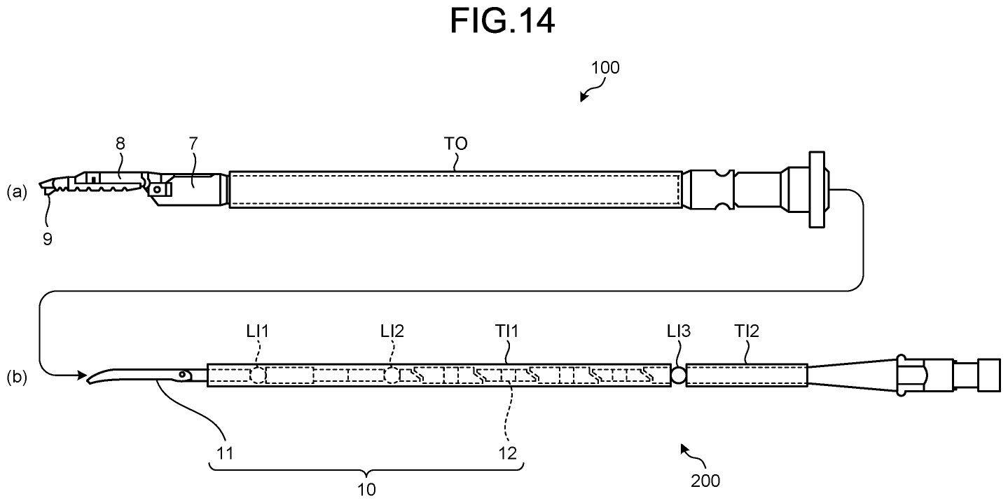

FIG. 14 to FIG. 16 are views for explaining Step S1.

Note that, hereinafter, for convenience of description, a unit in which the sheath 7, the outer tube TO, the jaw 8, and the resin pad 9 are integrated is referred to as a welding unit 100 (FIG. 14). Further, a unit in which the ultrasonic probe 10, the first to third linings LI1 to LI3, and the first and second inner tubes TI1 and TI2 are integrated is referred to as a probe unit 200 (FIG. 14).

First, the operator divides the holding case 4 in which the two bodies are combined into the two bodies. Then, the operator removes the welding unit 100 with the probe unit 200 inserted into the inner pipe 72 from the holding case 4.

Next, as illustrated in FIG. 14, the operator pulls out the probe unit 200 inserted into the inner pipe 72 from the proximal end of the sheath 7.

Next, the operator uses a cutter or the like to remove the first and second inner tubes TI1 and TI2 and the first to third linings LI1 to LI3 from the probe unit 200, as illustrated in FIG. 15. In the following, for convenience of description, the unit after the first and second inner tubes TI1 and TI2 and the first to third linings LI1 to LI3 are removed from the probe unit 200 is referred to as a reuse probe unit 201 (see FIG. 15(b)).

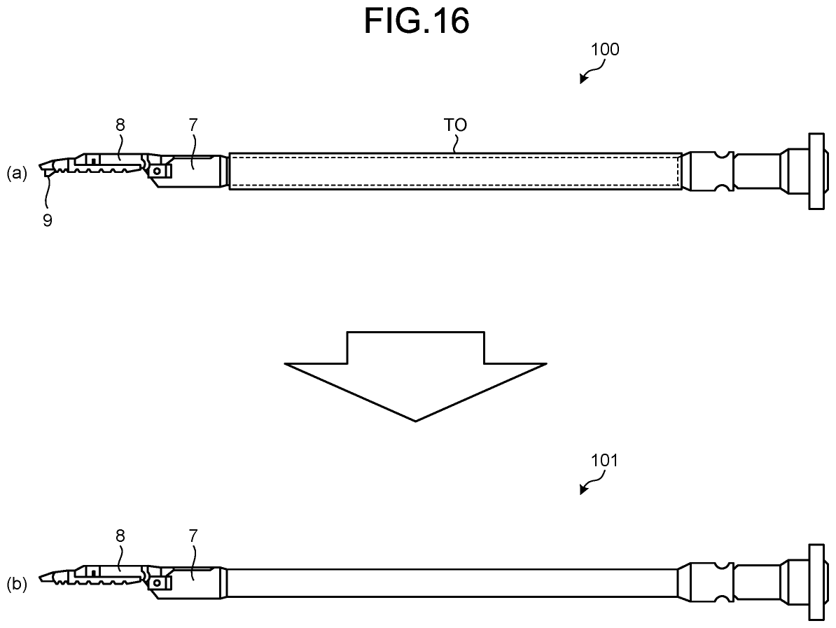

Next, the operator uses a cutter or the like to remove the outer tube TO from the welding unit 100, as illustrated in FIG. 16.

Next, the operator slides the pair of protruding portions 913 along the longitudinal direction of the wiper jaw 82 in the pair of grooves 832 to remove the resin pad 9 from the proximal end side Ar2 of the wiper jaw 82, for example. In the following, for convenience of description, the unit after the outer tube TO and the resin pad 9 are removed from the welding unit 100 is referred to as a reuse welding unit 101 (FIG. 16(b)).

After Step S1, the operator cleans, disinfects, and sterilizes the reuse probe unit 201 and the reuse welding unit 101 as described below (Step S2).

Specifically, in cleaning the reuse probe unit 201 and the reuse welding unit 101, a large amount of dirt adhering to the reuse probe unit 201 and the reuse welding unit 101 is removed by using a brush or the like. After that, in order to remove pathogenic microorganisms and the like derived from blood, body fluid, and mucosa, etc., ultrasonic cleaning is performed on the reuse probe unit 201 and the reuse welding unit 101 by using any one cleaning solution of isopropanol-containing detergent, proteolytic enzyme detergent, and alcohol. The cleaning solution is not limited to the above-described cleaning solution, and other cleaning solution may be adopted or disinfecting solution may be contained.

Further, in the sterilization of the reuse probe unit 201 and the reuse welding unit 101, in order to remove pathogenic microorganisms and the like derived from blood, body fluid, mucosa, etc., either one of high-pressure steam sterilization, ethylene oxide gas sterilization, and hydrogen peroxide gas low-temperature sterilization is used.

After Step S2, the operator assembles a new treatment instrument 1 as described below (Step S3).

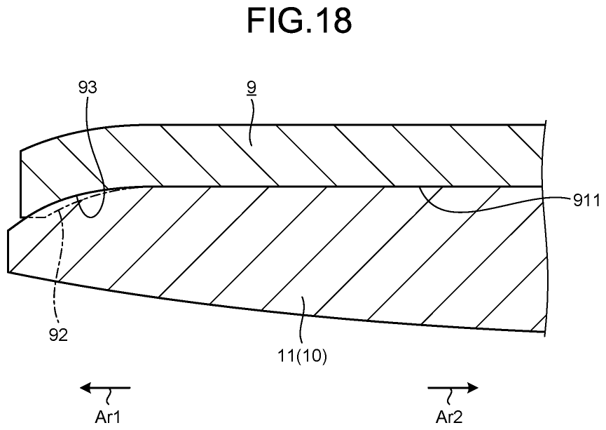

FIGS. 17A, 17B, and 18 are views for explaining Step S3. Specifically, FIG. 17A is a view illustrating a procedure of mounting the resin pad 9 to the wiper jaw 82, as seen along the width direction of the wiper jaw 82. FIG. 17B is a view illustrating the procedure of mounting the resin pad 9 to the wiper jaw 82, as seen from the blade side Ar4. FIG. 18 is a view illustrating a procedure of forming a depression 93 in the resin pad 9 by the blade 11.

First, the operator installs new first to third linings LI1 to LI3 in the reuse probe unit 201 at the respective positions P1 to P3 on the outer peripheral surface of the shaft 12. Further, the operator inserts the reuse probe unit 201 into a new second inner tube 112 from the blade 11 and positions the second inner tube 112 on the proximal end side Ar2 with respect to the third lining LI3. Then, the operator heat-shrinks the new second inner tube 112 by heating to bring the new second inner tube 112 into close contact with the outer peripheral surface of the shaft 12. Further, the operator inserts the reuse probe unit 201 into the new first inner tube TI1 from the blade 11 and positions the new first inner tube TI1 at a position where the new first inner tube TI1 covers the first and second linings LI1 and LI2.

Through the above steps, a new probe unit 200 is formed.

Next, the operator inserts the reuse welding unit 101 into a new outer tube TO from the jaw 8 and positions the new outer tube TO on the outer peripheral surface of the outer pipe 71. Then, the operator heat-shrinks the new outer tube TO by heating to bring the new outer tube TO into close contact with the outer peripheral surface of the outer pipe 71.

Next, as illustrated in FIG. 17A, the operator opens the jaw 8 toward the back surface side Ar3 in the reuse welding unit 101. Further, the operator slightly bends the distal end side Ar1 of a new resin pad 9 and, as illustrated in FIG. 17A or 17B, a pair of protruding portions 913 of the new resin pad 9 are inserted in the pair of grooves 832 from the proximal end of the wiper jaw 82. Then, the protruding portions 913 are slid toward the distal end side Ar1 in the pair of grooves 832, so that the new resin pad 9 is mounted to the wiper jaw 82. With this, the end portion of the new resin pad 9 on the distal end side Ar1 is located at the end portion of the wiper jaw 82 on the distal end side Ar1. Then, the new resin pad 9 is set by the bottom surface 831A of the second recessed portion 831 such that the end portion of the distal end side Ar1 is bent toward the blade side Ar4.

As described above, when the protruding portions 913 are slid toward the distal end side Ar1 in the pair of grooves 832, the blade side Ar4 of the second recessed portion 831 is preferably closed by using a jig or the like. By closing the blade side Ar4 of the second recessed portion 831 in this manner, the new resin pad 9 is prevented from protruding toward the blade side Ar4 from between the claws 831D adjacent to each other along the longitudinal direction of the wiper jaw 82.

Through the above steps, a new welding unit 100 is formed.

Next, the operator inserts the new probe unit 200 from the blade 11 into the inner pipe 72 of the new welding unit 100. Here, the two bodies obtained by dividing the holding case 4 in Step S1 are used again. Then, the operator installs the new welding unit 100 in a state in which the new probe unit 200 is inserted into the inner pipe 72 between the two bodies, and combines the two bodies to assemble a new treatment instrument 1.

Next, the operator operates the operation handle 5 in the new treatment instrument 1 to bring the jaw 8 close to the blade 11 to bring the resin pad 9 into contact with the blade 11. At this time, as described above, when the resin pad 9 is simply applied to the blade 11, a gap is formed between the inner surface of the third recessed portion 911 and the outer surface of the blade 11 due to the thick portion 92. Here, the resin pad 9 is softer than the blade 11. Therefore, when a predetermined pressing force is applied to the resin pad 9 from the blade 11, the region of the thick portion 92 on the proximal end side Ar2 is plastically deformed as illustrated in FIG. 18. The depression 93 (FIG. 18) having an inner surface shape complementary to the outer surface shape of the blade 11 is formed at the end portion of the resin pad 9 on the distal end side Ar1. That is, the formation of the depression 93 eliminates the above-described gap between the resin pad 9 and the blade 11. The region of the thick portion 92 on the distal end side Ar1 and the portion of the resin pad 9 other than the end portion on the distal end side Ar1 are not plastically deformed by the blade 11. That is, the region of the thick portion 92 on the distal end side Ar1 remains.

By forming the depression 93 as described above, the distal end of the resin pad 9 protrudes toward the blade 11 side. That is, the distal end of the resin pad 9 is formed in a claw shape.

Although the depression 93 is formed by plastic deformation in the above description, the present disclosure is not limited to this, and the depression 93 may be formed as described below.

The operator operates the operation handle 5 in the new treatment instrument 1 to bring the jaw 8 close to the blade 11 to bring the resin pad 9 into contact with the blade 11. After that, the operator presses the switch 6 to apply ultrasonic vibration from the blade 11 to the resin pad 9. Due to the ultrasonic vibration, frictional heat is generated between the blade 11 and the resin pad 9. As a result, the thick portion 92 is deformed. That is, the depression 93 is formed.

Step S3 described above corresponds to an assembly method for the treatment instrument 1 according to the present disclosure.

After Step S3, the operator performs an inspection and a test on the newly formed treatment instrument 1 as shown below (Step S4).

Specifically, as the inspection and test of the treatment instrument 1, various verification tests such as biocompatibility, cleaning validation, performance, EOG (ethylene oxide gas) sterilization residue test, bioburden resistance test, comparative resistance of sterilization, and viable cell count test can be exemplified. Here, with respect to the performance, it is verified that the newly formed treatment instrument 1 has effectiveness and safety equivalent to the original product.

After Step S4, the operator sequentially performs packaging (Step S5), box packing (Step S6), sterilization (Step S7), and shipping (Step S8) of the newly formed treatment instrument 1.

The treatment instrument 1 is remanufactured by Steps S1 to S8 described above.

According to the present embodiment described above, the following effects are achieved.

The resin pad 9 according to the present embodiment is provided with the thick portion 92. Then, by bringing the jaw 8 close to the blade 11 and pressing the blade 11 against the thick portion 92, the depression 93 having an inner surface shape complementary to the outer surface shape of the jaw 8 is formed at the end portion of the resin pad 9 on the distal end side Ar1. Then, by forming the depression 93, the distal end of the resin pad 9 is formed into a claw shape.

Therefore, even if there are variations in the manufacture of the resin pad 9, or variations in the relative position of the resin pad 9 with respect to the jaw 8 that occur when the resin pad 9 is assembled to the jaw 8, by forming the above-described depression 93, the resin pad 9 and the blade 11 can be brought into precise contact with each other. That is, the resin pad 9 and the blade 11 are properly meshed with each other, and the treatment performance of the target portion can be favorably maintained.

Further, the resin pad 9 according to the present embodiment is made attachable and detachable to and from the jaw 8 by sliding the pair of protruding portions 913 in the pair of grooves 832. Therefore, it is possible to easily remove the resin pad 9 from the jaw 8 in Step S1 and mount the resin pad 9 to the jaw 8 in Step S3.

### Other Embodiments

The embodiment for carrying out the present disclosure has been described above, but the present disclosure should not be limited only by the above-described embodiment.

In the above-described embodiment, the configuration in which both ultrasonic energy and high-frequency energy are applied to the target portion is adopted, but the present disclosure is not limited to this. For example, a configuration in which only ultrasonic energy is applied to the target portion, a configuration in which only high-frequency energy is applied to the target portion, a configuration in which only energy other than ultrasonic energy and high-frequency energy is applied to the target portion, or a configuration in which these configurations are combined may be adopted. Incidentally, when the configuration in which only ultrasonic energy is applied to the target portion is adopted, since it is not necessary to ensure electrical insulation between the outer pipe 71 and the inner pipe 72 and the ultrasonic probe 10, the outer tube TO and the first and second inner tubes TI1 and TI2 may be omitted.

In the above-described embodiment, the new resin pad 9 may be mounted to the jaw 8 at the following timing.

That is, the operator inserts the new probe unit 200 from the blade 11 into the inner pipe 72 of the reuse welding unit 101. Thereafter, the operator mounts the new resin pad 9 to the jaw 8 of the reuse welding unit 101 by the procedure illustrated in FIG. 17A.

In mounting the new resin pad 9 to the jaw 8, if the blade 11 is an obstruction, the new resin pad 9 may be inserted from not the proximal end of the wiper jaw 82 but the distal end of the wiper jaw 82 or between the claws 831D adjacent to each other along the longitudinal direction of the wiper jaw 82.

Although the first to third linings LI1 to LI3 and the second inner tube TI2 are removed from the probe unit 200 in Step S1 in the above-described embodiment, the present disclosure is not limited to this, and the first to third linings LI1 to LI3 and the second inner tube TI2 may be reused without being removed. Further, in the blade 11, when electrically insulating coating is applied to the outer surface of the back surface side away from the jaw 8, the coating may be removed in Step S1, or may be reused without being removed.

With the assembly method for a treatment instrument and the resin pad according to the present disclosure, the treatment performance can be favorably maintained.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An assembly method for an ultrasonic treatment instrument including: a first gripping member including an ultrasonic probe configured to treat a living tissue; and a second gripping member configured to open and close relative to the first gripping member, the assembly method comprising:
inserting a resin pad into a welding unit including a sheath, the resin pad including:
a pad body that includes a recessed portion and a thick portion provided at a distal end portion of the recessed portion,
connecting the second gripping member to the sheath along a groove provided in the second gripping member such that the recessed portion faces the first gripping member;
inserting the ultrasonic probe into the welding unit; and
forming a distal end of the resin pad into a claw shape by pressing the first gripping member against the thick portion by bringing the second gripping member and the first gripping member towards each other to form a depression having an inner surface shape complementary to an outer surface shape of the first gripping member at the thick portion.

2. The assembly method according to claim 1, including:
forming the depression in a region of the thick portion on a proximal end side; and
not forming the depression at a region of the thick portion on a distal end side of the second gripping member.

3. The assembly method according to claim 1, further including:
before forming the distal end of the resin pad into the claw shape, a shape of a surface of the resin pad that contacts the first gripping member does not complement the outer surface shape of the first gripping member due to the thick portion provided.

4. The assembly method according to claim 1, wherein the second gripping member includes:
an arm configured to open and close relative to the first gripping member, and
a wiper jaw axially supported on the arm, the wiper jaw including the groove, and
when inserting the resin pad, the distal end portion of the recessed portion is positioned at a distal end of the wiper jaw by inserting the resin pad along the groove.

5. The assembly method according to claim 1, wherein the resin pad is inserted after a used resin pad that has treated the living tissue is removed from the groove, and after cleaning and sterilization of the second gripping member.

6. The assembly method according to claim 1, including forming the depression by plastic deformation.

7. The assembly method according to claim 6, wherein the recessed portion is not formed by plastic deformation.

8. The assembly method according to claim 1, wherein
the first gripping member is configured to treat the living tissue by applying ultrasonic vibration to the living tissue, and
the depression is formed by applying ultrasonic vibration from the first gripping member to the thick portion.

9. An ultrasonic treatment instrument comprising:
a first gripping member configure to treat a living tissue by applying ultrasonic vibration;
a second gripping member co figured to open and close relative to the first gripping member; and
a resin pad provided on the second gripping member and extended from a distal end portion toward a proximal end portion, the resin pad including a thick portion at a distal end portion of the second gripping member,
wherein when the first gripping member is pressed against the thick portion by bringing the second gripping member and the first gripping member towards each other, the resin pad has a depression having an inner surface shape that complements an outer surface shape of the first gripping member, and a claw shape by the depression at a distal end side.

10. The ultrasonic treatment instrument according to claim 9, wherein, when the first gripping member is pressed against the thick portion by bringing the second gripping member and the first gripping member towards each other, the depression is formed in a region of the thick portion on a proximal end side, and a region of the thick portion on a distal end side remains.

11. The ultrasonic treatment instrument according to claim 9, wherein a shape of a surface of the resin pad that comes into contact with the first gripping member does not complement the outer surface shape of the first gripping member because of a position of the thick portion.

12. The ultrasonic treatment instrument according to claim 9,
wherein the resin pad includes a pad body having a protruding portion configured to be inserted into a groove provided in the second gripping member.

13. The ultrasonic treatment instrument according to claim 12, wherein the resin pad is configured to attach to or detach from the second gripping member by sliding the protruding portion in the groove.

14. The ultrasonic treatment instrument according to claim 9, wherein the resin pad is softer than the first gripping member.

15. The ultrasonic treatment instrument according to claim 9, wherein a shape of a recessed portion of the resin pad complements an outer surface shape of the first gripping member.

16. A method of re-manufacturing an ultrasonic treatment instrument including: a first gripping member including an ultrasonic probe configured to treat a living tissue; and a second gripping member configured to open and close relative to the first gripping member, the method comprising:
removing a welding unit including a sheath and the second gripping member connected to the sheath from the ultrasonic treatment instrument by disassembling the ultrasonic treatment instrument;
cleaning, disinfecting, or sterilizing the ultrasonic probe and the welding unit;
inserting a resin pad, including a pad body, into the welding unit along a groove provided in the second gripping member, the pad body including a recessed portion and a thick portion at a distal end portion of the recessed portion, such that, when inserted, the recessed portion faces the first gripping member;
inserting the ultrasonic probe into the welding unit; and
forming a distal end of the resin pad into a claw shape by pressing the first gripping member against the thick portion by bringing the second gripping member and the first gripping member towards each other to form a depression having an inner surface shape that complements an outer surface shape of the first gripping member at the thick portion.
